(19) United States
(12) Patent Application Publication
(10) Pub. No.: US 2020/0323484 A1
(43) Pub. Date: Oct. 15, 2020

(54) METHOD AND SYSTEM FOR SCREENING FOR COVID-19 WITH A VOCAL BIOMARKER

(71) Applicant: VOCALIS HEALTH, Tel-Aviv (IL)

(72) Inventors: Daniel ARONOVICH, Haifa (IL); Shadi HASSAN, Julis (IL)

(21) Appl. No.: 16/906,091

(22) Filed: Jun. 19, 2020

Related U.S. Application Data
(63) Continuation-in-part of application No. 16/218,878, filed on Dec. 13, 2018.
(60) Provisional application No. 63/040,584, filed on Jun. 18, 2020.
(60) Provisional application No. 62/598,477, filed on Dec. 14, 2017.

(51) Int. Cl. A61B 5/00; G10L 19/26; G10L 25/30; G10L 25/66; G10L 17/02; G10L 17/18

(52) U.S. Cl. CPC A61B 5/4803; A61B 5/749; A61B 5/7257; A61B 5/7267; G10L 17/18; G10L 25/30; G10L 25/66; G10L 17/02; G10L 19/26

(57) ABSTRACT

A computer-based method for screening unknown subjects for COVID-19, including steps of recording at least one voice clip from a screened subject, pre-processing the screened subject voice clip, computing a spectrogram of the pre-processed screened subject voice clip, extracting a feature vector from said screened subject spectrogram, applying a machine learning classifier of a COVID-19 vocal biomarker on the extracted screened subject feature vector thereby receiving a COVID-19 vocal biomarker value, and outputting that the screened subject is COVID-19 positive or COVID-19 negative, based on the COVID-19 vocal biomarker value. The step of extracting the feature vector employs a pre-trained deep convolutional neural network.

### CROSS REFERENCE TO RELATED APPLICATIONS

This application is a continuation-in-part of non-provisional application Ser. No. 16/218,878 filed on Dec. 13, 2018, which claims the benefit of and priority to 62/598,477 filed on Dec. 14, 2017. The contents of these applications are incorporated by reference in their entirety.

The present application additionally claims the priority benefit of and priority to U.S. Provisional application No. 63/040,584, filed on Jun. 18, 2020. The content of this application is incorporated herein in its entirety.

### FIELD OF THE INVENTION

The invention is in the field of voice analysis, and in particular to a system and method for providing a vocal biomarker for COVID-19 screening.

### BACKGROUND OF THE INVENTION

The COVID-19 pandemic is a major challenge for governments, businesses, healthcare systems and people around the globe seeking ways to safely return to work/healthcare/travel/leisure. Testing for this highly infectious and often asymptomatic disease is burdensome with limited availability; treatments and vaccines are as yet unproven.

On Mar. 11, 2020, the World Health Organization declared the coronavirus disease (COVID-19) outbreak a pandemic. Since the disease was first reported in late December 2019 in Wuhan, China, it has spread to more than 216 countries and territories globally. As of 9 Jun. 2020, U.S. Pat. No. 7,039,918 confirmed cases and 404,396 deaths have been reported worldwide [World Health Organization]. Isolation strategies have slowed the transmission but caused major disruptive changes to everyday life and led to an economic recession.

COVID-19 infection causes clusters of respiratory illness and is associated with intensive care unit admission and high mortality rates, especially in older persons, or populations with underlying illness. Common symptoms include fever, cough, shortness of breath and myalgia or fatigue, but symptoms vary dramatically between patients and the majority of infected patients are asymptomatic [Rothe, Yu, Bai].

The present invention provides a method and system for screening subjects with COVID-19.

### SUMMARY OF THE INVENTION

The present invention relates to a vocal biomarker for COVID-19 screening. The COVID-19 vocal biomarker can enable a return to normal activities in the context of ongoing risk of COVID-19 infection.

Current diagnostic testing methodologies such as polymerase chain reaction-based methods or deep sequencing play an indispensable role, but have rigorous laboratory specifications and results are not available immediately. These methods also rely on the presence of sufficient viral load at the site of sample collection, creating the potential for false-negative results [Guo, L].

Body temperature screening is the main test performed to screen risk at points of entry (i.e., in healthcare settings, factories, airports, retail). Two studies summarizing data of 1,099 and 5,700 admitted patients with laboratory confirmed COVID-19 in China and the New York City area reported that only 43.8% and 30.7% of patients had fever on admission, respectively.7,8 Recent reports on asymptomatic contact transmission of COVID-19 and false-negative results of symptom-based screening challenge this approach as fever screening may miss individuals incubating the disease [Buire].

Voice is a non-invasive, passive signal that can serve as a biomarker to screen and monitor health. Voice analysis has been used to detect Parkinson's Disease, obstructive sleep apnea and autism spectrum disorder [Bonneh, Uma, Goldshtein].

It is therefore within the scope of the invention to provide a computer-based method for screening unknown subjects for COVID-19, comprising steps of
a) recording at least one voice clip from a screened subject;
b) pre-processing the screened subject voice clip;
c) computing a spectrogram of the pre-processed screened subject voice clip;
d) extracting a feature vector from said screened subject spectrogram;
e) applying a machine learning classifier of a COVID-19 vocal biomarker on the extracted screened subject feature vector, thereby receiving a COVID-19 vocal biomarker value; and
f) outputting that the screened subject is COVID-19 positive or COVID-19 negative, based on the COVID-19 vocal biomarker value;
wherein the step of extracting the feature vector employs a pre-trained deep convolutional neural network (CNN).

It is further within the scope of the invention to provide the abovementioned method, wherein recording said at least one voice clip is made at a sampling rate of 16 kHz, 32 kHz, 44.1 kHz.

It is further within the scope of the invention to provide any one of the abovementioned methods, further comprising selecting one or more of the speech clips from a fixed time interval of continuous speech within an extended recording of one or more of the subjects.

It is further within the scope of the invention to provide the previous method, wherein the fixed time interval is 10 seconds.

It is further within the scope of the invention to provide any one of the abovementioned methods, wherein the pre-processing of said at least one voice clip comprises one or more steps selected from a group consisting of normalizing, down-sampling, and any combination thereof.

It is further within the scope of the invention to provide the previous method, wherein the down-sampling is made to 16 kHz.

It is further within the scope of the invention to provide any one of the abovementioned methods, wherein the computing of the spectrograms is made with an algorithm selected from a group comprising a short-time Fourier transform (STFT), a fast Fourier transform (FFT), Mel spectrogram, or any combination thereof.

It is further within the scope of the invention to provide any one of the abovementioned methods, wherein the feature vectors each comprise 512 or 1024 dimensions.

It is further within the scope of the invention to provide any one of the abovementioned methods, wherein said at least one voice clip comprises scripted speech, free speech, or any combination thereof.

It is further within the scope of the invention to provide any one of the abovementioned methods, further comprising steps for training said COVID-19 vocal biomarker, comprising
a) recording at least one voice clip from each subject in a cohort, each cohort subject having a known status of either COVID-19 positive and COVID-19;
b) pre-processing the cohort subject voice clips;
c) computing a spectrogram of each of the pre-processed cohort subject voice clips;
d) extracting feature vectors from each cohort subject spectrogram, using said CNN; and
e) training a machine classifier with said cohort subject feature vectors and said cohort subjects' known COVID-19 statuses, thereby producing said COVID-19 vocal biomarker.

It is further within the scope of the invention to provide the previous screening and training method, further comprising a step of cross-validating models for developing said classifier.

It is further within the scope of the invention to provide the previous screening and training method, wherein the cross-validating is 10-fold.

It is further within the scope of the invention to provide any one of the previous two screening and training methods, further comprising a step of selecting one or more of said models with the highest areas-under-curve (AUCs) of a receiver operating curve (ROC) of each cross-validated model.

It is further within the scope of the invention to provide any one of the abovementioned screening and training methods, further comprising a step of selecting an equal number of COVID-19 positive subjects and COVID-19 negative subjects for said cohort.

It is further within the scope of the invention to provide the previous screening and training method, further comprising a step of pairing each of the COVID-19 positive subjects with one of the COVID-19 negative subjects who speaks the same language, has the same gender, a has a similar age

It is further within the scope of the invention to provide the previous screening and training method, wherein the similar age is defined as within one year.

It is further within the scope of the invention to provide any one of the previous screening and training methods, wherein the cohort subject voice clips comprise both scripted speech clips and free speech clips.

It is further within the scope of the invention to provide any one of the previous screening and training methods, further comprising a step of dividing said cohort into two groups: subjects with a fever and subjects with no fever; and wherein the cross-correlating is independently verified for both groups.

It is further within the scope of the invention to provide a computer-based system for screening unknown subjects for COVID-19, comprising
a) a recording module, configured to record at least one voice clip from a screened subject;
b) a pre-processing module, configured to pre-process the screened subject voice clip;
c) a spectrography module, configured to compute a spectrogram of the pre-processed screened subject voice clip;
d) a feature-extraction module, configured to extract a feature vector from said screened subject spectrogram;
e) a classification module, configured to apply a machine learning classifier of a COVID-19 vocal biomarker on the extracted screened subject feature vector, thereby receiving a COVID-19 vocal biomarker value; and
f) an output module, configured to output that the screened subject is COVID-19 positive or COVID-19 negative, based on the COVID-19 vocal biomarker value;
   wherein the step of extracting the feature vector employs a pre-trained deep convolutional neural network (CNN).

It is further within the scope of the invention to provide the abovementioned system, wherein recording said at least one voice clip is made at a sampling rate of 16 kHz, 32 kHz, 44.1 kHz.

It is further within the scope of the invention to provide any one of the abovementioned systems, wherein the recording module is further configured to select one or more of the speech clips from a fixed time interval of continuous speech within an extended recording of one or more of the subjects.

It is further within the scope of the invention to provide the previous system, wherein the fixed time interval is 10 seconds.

It is further within the scope of the invention to provide any one of the abovementioned systems, wherein the pre-processing is further configured to pre-process by normalizing, down-sampling, or any combination thereof.

It is further within the scope of the invention to provide the previous system, wherein the down-sampling is made to 16 kHz.

It is further within the scope of the invention to provide any one of the abovementioned systems, wherein the computing of the spectrograms is made with an algorithm selected from a group comprising a short-time Fourier transform (STFT), a fast Fourier transform (FFT), Mel spectrogram, or any combination thereof.

It is further within the scope of the invention to provide any one of the abovementioned systems, wherein the feature vectors each comprise 512 or 1024 dimensions.

It is further within the scope of the invention to provide any one of the abovementioned systems, wherein said at least one voice clip comprises scripted speech, free speech, or any combination thereof.

It is further within the scope of the invention to provide any one of the abovementioned systems, further comprising a training module for training said COVID-19 vocal biomarker, said training module configured to
a) record at least one voice clip from each subject in a cohort, each cohort subject having a known status of either COVID-19 positive and COVID-19;
b) pre-process the cohort subject voice clips;
c) compute a spectrogram of each of the pre-processed cohort subject voice clips;
d) extract feature vectors from each cohort subject spectrogram, using said CNN; and
e) train a machine classifier with said cohort subject feature vectors and said cohort subjects' known COVID-19 statuses, thereby producing said COVID-19 vocal biomarker.

It is further within the scope of the invention to provide the abovementioned training and screening system, further configured to cross-validate models for developing said classifier.

It is further within the scope of the invention to provide the previous training and screening system, wherein the cross-validating is 10-fold.

It is further within the scope of the invention to provide any one of the abovementioned training and screening systems, wherein the training module is further configured to select one or more of said models with the highest areas-under-curve (AUCs) of a receiver operating curve (ROC) of each cross-validated model.

It is further within the scope of the invention to provide any one of the abovementioned training and screening systems, wherein the training module is further configured to select an equal number of COVID-19 positive subjects and COVID-19 negative subjects for said cohort.

It is further within the scope of the invention to provide the previous training and screening system, wherein the training module is further configured to pair each of the COVID-19 positive subjects with one of the COVID-19 negative subjects who speaks the same language, has the same gender, a has a similar age.

It is further within the scope of the invention to provide the previous training and screening system, wherein the similar age is defined as within one year.

It is further within the scope of the invention to provide any one of the abovementioned training and screening systems, wherein the cohort subject voice clips comprise both scripted speech clips and free speech clips.

It is further within the scope of the invention to provide any one of the abovementioned training and screening systems, wherein the training module is further configured to comprising divide said cohort into two groups: subjects with a fever and subjects with no fever; and wherein the cross-correlating is independently verified for both groups.

In some embodiments, the teachings of the parent application (for a method and system for diagnosing coronary artery disease) apply as well to screening subjects for COVID-19. These teachings—including systems, methods, features, and technical details—may constitute one or more embodiments of the present invention, in whole or in part. Some of these embodiments are now described:

A computer-implemented second method for screening a subject for COVID-19, comprising: receiving voice signal data indicative of speech from the patient; segmenting the voice signal data into frames of, for example, 32 ms with a frame shift of 10 ms; applying Mel Frequency Cepstral Coefficients (MFCC) module; computing a Cepstral representation using cosine transform; determining a COVID-19 status of the subject; Mel Frequency Cepstral Coefficients (MFCC) module is applied by assigning type operator functions across the one or more of frequencies on one or more sample intensity values of the voice signal data; and a COVID-19 status of the subject is determined based at least in part upon a change in intensity between at least two frequencies found in the Cepstral representation and/or calculated type operator function.

The abovementioned second method, wherein the step of computing MFCC is performed by computing a Cepstral representation using any degree of freedom.

Any one of the abovementioned second methods, wherein the Cepstral representation comprises time-series is used for statistical feature extraction.

Any one of the abovementioned second methods, wherein the step of segmenting the voice signal data into frames, further provides a power spectrum density (PSD) and/or its Root Mean Squaring (RMS) spectrogram with any resolution between 1 to 200 frames per second.

Any one of the abovementioned second methods, wherein the step of computing Mel Frequency Cepstral Coefficients (MFCC) from a log scaling function that resemble the human acoustic perception of sounds is achieved by using any number of Mel frequency triangular filter banks.

Any one of the abovementioned second methods, wherein the step of computing Mel Frequency Cepstral Coefficients (MFCC) from a log scaling functions that resemble the human acoustic perception of sound pressure levels is achieved by converting to decibels (DB).

Any one of the abovementioned methods, wherein for each of the two or more of frequency bands the intensity ratio values is manifested at a given time period.

Any one of the abovementioned second methods, wherein the voice signal data has a finite duration and each time period separating the respective plurality of intensity ratio values is essentially evenly distributed within the duration of the speech.

Any one of the abovementioned second methods, wherein the COVID-19 status of a subject is determined based at least in part upon the type of statistical operator function including at least one decay feature.

The previous second method, wherein the zero-crossing type operator measure provides an indicator of the severity of the COVID-19.

Any one of the two previous second methods, wherein the averaging type operator measure provides an indicator of the severity of the COVID-19.

Any one of the previous three second methods, wherein the maximum type operator measure provides an indicator of the severity of the COVID-19.

Any one of the previous four second methods, at least one of a height and a width of the crater feature provides an indicator of the severity of the COVID-19.

Any one of the abovementioned second methods, wherein the COVID-19 status of a subject is determined based at least in part upon the zero-crossing and/or averaging and/or maximum statistical operators including at least one crater feature.

A computer-implemented second system for screening a subject for COVID-19, the system comprising: one or more processors; and a memory system communicatively coupled to the one or more processors, the memory system comprises executable instructions including: receiving voice signal data indicative of speech from the patient; segmenting the voice signal data into frames of 32 ms with a frame shift of 10 ms; applying Mel Frequency Cepstral Coefficients (MFCC) module; computing a Cepstral representation using cosine transform; determining a COVID-19 status of the subject; Mel Frequency Cepstral Coefficients (MFCC) module is applied by assigning type operator functions across the one or more of frequencies on one or more sample intensity values of the voice signal data; and a COVID-19 status of the subject is determined based at least in part upon a change in intensity between at least two frequencies found in the Cepstral representation and/or calculated type operator function.

The abovementioned second system, wherein the step of computing MFCC is performed by computing a Cepstral representation using any degree of freedom.

Any one of the abovementioned second systems, wherein the Cepstral representation comprises time-series is used for statistical feature extraction.

Any one of the abovementioned second systems, wherein the step of segmenting the voice signal data into frames, further provides a power spectrum density (PSD) and/or its Root Mean Squaring (RMS) spectrogram with any resolution between 1 to 200 frames per second.

Any one of the abovementioned second systems, wherein the step of computing Mel Frequency Cepstral Coefficients (MFCC) from a log scaling function that resemble the human acoustic perception of sounds is achieved by using any number of Mel frequency triangular filter banks.

Any one of the abovementioned second systems, wherein the step of computing Mel Frequency Cepstral Coefficients (MFCC) from a log scaling functions that resemble the human acoustic perception of sound pressure levels is achieved by converting to decibels (DB).

Any one of the abovementioned second systems, wherein for each of the two or more of frequency bands the intensity ratio values is manifested at a given time period.

Any one of the abovementioned second systems, wherein the voice signal data has a finite duration and each time period separating the respective plurality of intensity ratio values is essentially evenly distributed within the duration of the speech.

Any one of the abovementioned second systems, wherein the COVID-19 status of a subject is determined based at least in part upon the type of statistical operator function including at least one decay feature.

The previous second second system, wherein the zero-crossing type operator measure provides an indicator of the severity of the COVID-19.

Any one of the previous two second systems, wherein the averaging type operator measure provides an indicator of the severity of the COVID-19.

Any one of the previous three second systems, wherein the maximum type operator measure provides an indicator of the severity of the COVID-19.

Any one of the previous four second systems, wherein at least one of a height and a width of the crater feature provides an indicator of the severity of the COVID-19.

Any one of the abovementioned second systems, wherein the COVID-19 status of a subject is determined based at least in part upon the zero-crossing and/or averaging and/or maximum statistical operators including at least one crater feature.

The details of one or more embodiments are set forth in the accompanying drawings and the description below. Other features, objects, and advantages of the invention will be apparent from the description and drawings, and from the claims.

### BRIEF DESCRIPTION OF THE DRAWINGS

FIG. 1 shows a list of steps of a method for training a COVID-19 vocal biomarker, according to some embodiments of the invention.

FIG. 2 shows system for training and screening subjects with a COVID-19 vocal biomarker, according to some embodiments of the invention.

FIG. 3 illustrates the feature extraction process conducted in each analysis using transfer learning and adaptation methods.

FIG. 4 shows an ROC curve of an optimal model for a COVID-19 biomarker, according to some embodiments of the invention.

FIG. 5 shows the predictive accuracy of the COVID-19 biomarker and compares it to the reported presence of COVID-19 in a free speech test set.

FIG. 6 shows the predictive accuracy of the COVID-19 biomarker and compares it to COVID-19 positivity on the symptoms test set.

### DETAILED DESCRIPTION OF THE PREFERRED EMBODIMENTS

In the following detailed description of the preferred embodiments, reference is made to the accompanying drawings that form a part hereof, and in which are shown by way of illustration specific embodiments in which the invention may be practiced. It is understood that other embodiments may be utilized and structural changes may be made without departing from the scope of the present invention. The present invention may be practiced according to the claims without some or all of these specific details. For the purpose of clarity, technical material that is known in the technical fields related to the invention has not been described in detail so that the present invention is not unnecessarily obscured.

While the technology will be described in conjunction with various embodiment(s), it will be understood that they are not intended to limit the present technology to these embodiments. On the contrary, the present technology is intended to cover alternatives, modifications and equivalents, which may be included within the spirit and scope of the various embodiments as defined by the appended claims.

Furthermore, in the following description of embodiments, numerous specific details are set forth in order to provide a thorough understanding of the present technology. However, the present technology may be practiced without these specific details. In other instances, well known methods, procedures, components, and circuits have not been described in detail as not to unnecessarily obscure aspects of the present embodiments.

Unless specifically stated otherwise as apparent from the following discussions, it is appreciated that throughout the present description of embodiments, discussions utilizing terms such as “computing”, “detecting,” “calculating”, “processing”, “performing,” “identifying,” “determining” or the like, refer to the actions and processes of a computer system, or similar electronic computing device. The computer system or similar electronic computing device manipulates and transforms data represented as physical (electronic) quantities within the computer system's registers and memories into other data similarly represented as physical quantities within the computer system memories or registers or other such information storage, transmission, or display devices, including integrated circuits down to and including chip level firmware, assembler, and hardware based micro code.

While the invention is susceptible to various modifications and alternative forms, specific embodiments thereof have been shown by way of example in the drawings and the above detailed description. It should be understood, however, that it is not intended to limit the invention to the particular forms disclosed, but on the contrary, the intention is to cover all modifications, equivalents, and alternatives falling within the spirit and scope of the invention as defined by the appended claims.

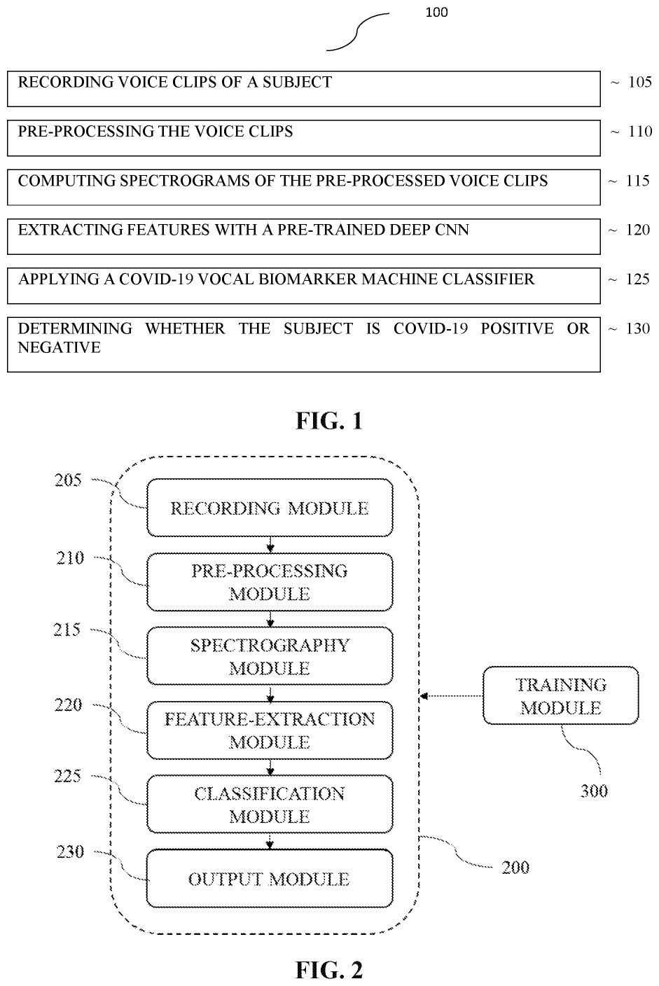

Reference is now made to FIG. 1, showing a list of steps of a computer-based method 100 for screening unknown subjects for COVID-19, comprising steps of

a) recording at least one voice clip from a screened subject 105;
b) pre-processing the screened subject voice clip 110;
c) computing a spectrogram of the pre-processed screened subject voice clip 115;
d) extracting a feature vector from said screened subject spectrogram 120;
e) applying a machine learning classifier of a COVID-19 vocal biomarker on the extracted screened subject feature vector, thereby receiving a COVID-19 vocal biomarker value 125; and
f) outputting that the screened subject is COVID-19 positive or COVID-19 negative, based on the COVID-19 vocal biomarker value 130;
wherein the step of extracting the feature vector employs a pre-trained deep convolutional neural network (CNN).

Reference is now made to FIG. 2, showing a system 200 A computer-based system for screening unknown subjects for COVID-19, comprising

a) a recording module 205, configured to record at least one voice clip from a screened subject;
b) a pre-processing module 210, configured to pre-process the screened subject voice clip;
c) a spectrography module 115, configured to compute a spectrogram of the pre-processed screened subject voice clip;
d) a feature-extraction module 210, configured to extract a feature vector from said screened subject spectrogram;
e) a classification module 225, configured to apply a machine learning classifier of a COVID-19 vocal biomarker on the extracted screened subject feature vector, thereby receiving a COVID-19 vocal biomarker value; and
f) an output module 130, configured to output that the screened subject is COVID-19 positive or COVID-19 negative, based on the COVID-19 vocal biomarker value;
wherein the step of extracting the feature vector employs a pre-trained deep convolutional neural network (CNN).

Details of methodologies used to train and test a system for screening subjects exhibiting a COVID-19 biomarker, according to some embodiments of the invention, are now described.

### Data Collection

Data on 953 participants were collected in parallel using three methods and used as described herein to establish a vocal biomarker for COVID-19:
Clinical trial (n=49). A prospective, multicenter, observational clinical study included patients with a positive COVID-19 test, cared for by medical staff members with a negative COVID-19 test. Centers included the Sheba Tel-Hashomer hospital (n=9), Rabin Medical Center (n=6) and the Israeli Defense Force (Israeli Army) personnel (n=34) in Israel. Participants signed informed consent; demographic and medical data were documented by research coordinators.
Online survey (n=578). A large-scale, crowdsourced data collection effort used an online, open call to participants who were either healthy or diagnosed with COVID-19 to join the study. Participants documented their demographic and medical data using a research mobile phone application developed by Vocalis Health.
YouTube audio (n=326). An active search online was conducted for interviews of individuals diagnosed with COVID-19 as well as healthy individuals on YouTube.

All participants recorded their voice and completed a symptom questionnaire on their smartphone, personal computer or tablet.

### Pre-Processing and Analysis

Because the data included recordings from an online survey, quality tests were done to ensure recording quality. For the analysis, 578 high-quality recordings from the online survey were included, out of 977 online survey recordings collected; 399 were excluded from the analysis because they were unclear, too short or noisy, or did not follow instructions. All recordings from the clinical trial and YouTube analysis were included. All recordings were sampled at a frequency of 44.1 kHz and normalized between a range of −1 and 1. The first 10 seconds of continuous speech in each recording were used in the analysis.

For the analyses described in this paper, the data were divided into a training set and a test set for each analysis. In each analysis, the same voice feature extraction and model evaluation processes described below were conducted on the training set, and a validation procedure was performed on the test set. The results of the biomarker performance are described in detail at the end of each analysis.

### Voice Feature Extraction

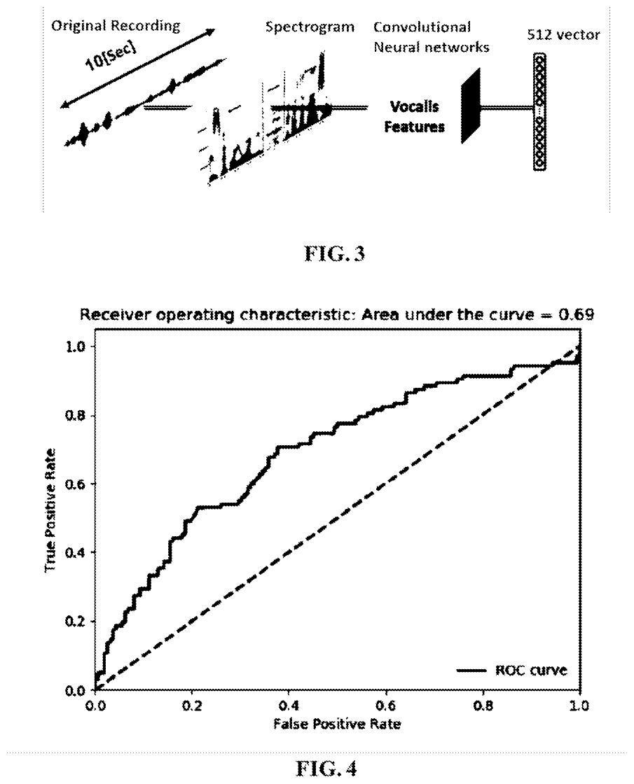

Reference is now made to FIG. 3, illustrating the feature extraction process conducted in each analysis using transfer learning and adaptation methods. The feature extraction process was based on published transfer learning and adaptation methods [Kumar]. Recordings were down-sampled to 16 kHz and a spectrogram was computed using the Short-Time Fourier Transform. Each spectrogram was passed through a pre-trained deep convolutional neural network, which resulted in a 512-dimensional features vector for each recording. This approach enables state-of-the-art results with small training databases.

### Biomarker Training and Model Evaluation Process

A 10-fold cross validation procedure was conducted, and several models were evaluated (k-nearest neighbours, support vector machine and random forest) at different regularization levels. In each analysis, the results of the models were evaluated by the average area under the receiver operating curve (AUC). The model selected for each analysis is described as the vocal biomarker, a positive scalar between 0-1, which is a non-linear combination of the 512 features mentioned above.

### Analysis 1. Assessing the COVID-19 Vocal Biomarker Performance on a Balanced Dataset

Of n=953 participants, 8% of participants were COVID-19 positive (n=78) and 92% were COVID-19 negative (n=875). A balanced training set was constructed in which each positive participant was paired with a negative participant who spoke the same language, had the same gender and a similar age (no more than a 1-year difference). This training set included a total of 156 participants from all three methods of data collection. It is important to note that some of the recordings contained scripted speech (clinical trial, online survey) while others contained free speech (YouTube). Baseline characteristics of the balanced training set are summarized in Table 1.

TABLE 1
Baseline characteristics of the balanced training set

| | Total | Positive | Negative |
|---|---|---|---|
| | Participants | COVID-19 | COVID-19 |
| | (n = 156) | (N = 78) | (n = 78) |
| Age (years) | 35 ± 14 | 35.6 ± 14.2 | 34.3 ± 14 |
| Male (%) | 108 (70%) | 54 (70%) | 54 (70%) |
| Language: | | | |
| Hebrew | 64 (41%) | 32 (41%) | 32 (41%) |
| English | 92 (59%) | 46 (59%) | 46 (59%) |
| Study method: | | | |
| Clinical trial | 49 (31%) | 32 (41%) | 17 (22%) |
| Other | 107 (69%) | 46 (59%) | 61 (78%) |

Note:
COVID-19 confirmed by PCR in the Clinical Trial group and self-reported in the other groups

The feature extraction and clinical validation procedures described above were performed on each recording in the balanced training set. The optimal result of our 10-fold procedure was an AUC of 0.69, using a support vector machine model with a nonlinear kernel (radial basis function). FIG. 4 shows the receiver operating curve. For further clinical validation, we randomized the labels (positive/negative) between the recordings and identified an AUC 0.5-0.53, which is equal to a random classifier. This test validated there was no data leakage of outside data into the training set and vice versa.

### Analysis 2. Assessing the COVID-19 Vocal Biomarker Performance on a Free Speech Dataset

In order to evaluate the capability of the biomarker to operate on free speech, we created a new training set with the exclusion of the COVID-19 positive YouTube recordings (n=27) from the training set described in the first analysis. The new training set included a selected subset containing 129 recordings (one recording per participant) which contained only scripted speech (either counting or reciting a predefined phrase). The characteristics of the new training set can be seen in Table 2.

TABLE 2
Baseline characteristics of the scripted speech training set.

| | Total | Positive | Negative |
|---|---|---|---|
| | participants | COVID-19 | COVID-19 |
| | (n = 129) | (N = 51) | (n = 78) |
| Age (years) | 33.8 ± 13.7 | 33 ± 13 | 34.34 ± 14 |
| Male (%) | 93 (72%) | 39 (76%) | 54 (69%) |
| Language: | | | |
| Hebrew | 49 (38%) | 32 (63%) | 17 (22%) |
| English | 80 (62%) | 19 (37%) | 61 (78%) |
| Study method: | | | |
| Clinical trial | 49 (38%) | 32 (63%) | 17 (22) |
| Online survey | 80 (62%) | 19 (37%) | 61 (78%) |

Note:
COVID-19 status confirmed by PCR in the Clinical Trial group and self-reported in the Other groups

The free speech test-set was comprised of 326 YouTube audio clips (a single clip/individual), all containing free speech. Twenty-seven individuals were self-described as positive for COVID-19 and 299 were COVID-19 negative. The clips of the negative group were recorded prior to the end of 2018, approximately 1 year before the first reports of COVID-19. All audio clips were traced by the Vocalis Health team of labelers, who also labeled their quality and assured that the interviewee was either positive for COVID-19 (based on the content of the interview) or negative (based on the date of recording). The age and gender of participants in this test set were calculated using the Vocalis Health classifier which was previously trained on 200,000 samples and tested on a hold-out set of 2,800 mutually exclusive samples, reaching an accuracy of 94% for age classification and 99.5% for gender. The baseline characteristics of the free speech test set are summarized in Table 3.

TABLE 3
Baseline characteristics of the free speech test set

| | Total | Positive | Negative |
|---|---|---|---|
| | participants | COVID-19 | COVID-19 |
| | (n = 326) | (N = 27) | (n = 299) |
| Age (years) | 27 ± 10 | 31 ± 15 | 27 ± 10 |
| Male (%) | 201 (62%) | 15 (55%) | 186 (62%) |
| Language: | | | |
| Hebrew | 14 (4%) | 14 (52%) | 0 (0%) |
| English | 312 (96%) | 13 (48%) | 299 (100%) |

Note:
COVID-19 confirmed by PCR in the Clinical Trial group and self-reported in the other groups

As described above, the first 10 seconds of continuous speech in each recording were used in the feature extraction process followed by the support vector machine classifier that was optimized on the scripted speech training set (Table 2). A threshold of 0.5 was chosen, meaning that each recording with a result above 0.5 was labelled as positive. The biomarker predictions were compared to the COVID-19 labels (FIG. 3). The sensitivity of the biomarker was 51.8% [95% CI: 32-71%], the specificity was 78.3% [95% CI: 73-883%], positive predictive value was 17.7% [95% CI: 12.3-24.7%] and negative predictive value was 94.7% [95% CI: 92.4-96.4%].

In order to verify that the biomarker is language agnostic, we performed a sub-analysis which included only the English speakers from the positive group (n=13).

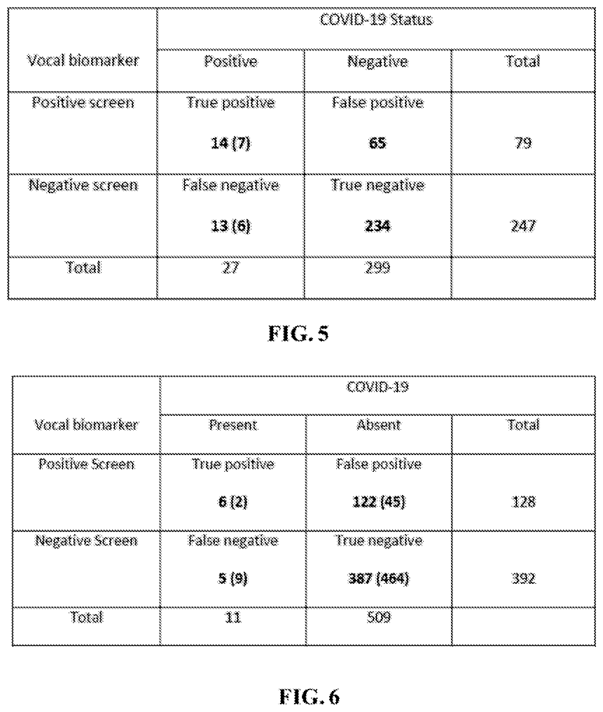

Reference is now made to FIG. 5. The predictive accuracy of the Vocalis COVID-19 biomarker is shown and compared to the reported presence of COVID-19 in the free speech test set. The sensitivity of the biomarker was 51.8% [95% CI 32-71%], specificity 78.3% [95% CI: 73-883%], positive predictive value 17.7% [95% CI: 12.3-24.7%]. The negative predictive value 94.7% [95% CI: 92.4-96.4%].

The results of the analysis matched the results of the entire group (English and Hebrew) as can be seen in parentheses in FIG. 5.

Analysis 3. Assessing the COVID-19 Vocal Biomarker Performance Vs. Fever Screening

Participants' symptoms were captured in the online survey dataset to compare the performance of the biomarker to fever, the most common symptom used in screening. A new training set and test set were created. Online survey recordings (n=22) from the training set described in the first analysis were excluded. The new training set included 134 recordings (one recording per participant), which contained scripted or free speech. The characteristics of the training set are noted in Table 4.

TABLE 4
Baseline characteristics of the symptoms training set.

| | Total | Positive | Negative |
|---|---|---|---|
| | participants | COVID-19 | COVID-19 |
| | (n = 134) | (N = 67) | (n = 67) |
| Age (years) | 34 ± 14.4 | 36 ± 14.7 | 32.6 ± 14 |
| Male (%) | 94 (70%) | 47 (70%) | 47 (70%) |
| Language: | | | |
| Hebrew | 34 (22%) | 17 (22%) | 17 (25%) |
| English | 100 (78%) | 50 (78%) | 50 (75%) |
| Study method: | | | |
| Clinical trial | 49 (36%) | 17 (22%) | 32 (48) |
| Online survey | 85 (64%) | 50 (78%) | 35 (52%) |

Note:
COVID-19 confirmed by PCR in the Clinical Trial group and self-reported in the other groups

The new test set included all recordings from the online survey (N=520); 11 participants in the test set were positive for COVID-19 and 509 were negative (Table 5).

TABLE 5
Baseline characteristics of the symptoms test set.

| | Total | Positive | Negative |
|---|---|---|---|
| | participants | COVID-19 | COVID-19 |
| | (n = 520) | (N = 11) | (n = 509) |
| Age (years) | 37 ± 14.3 | 34 ± 8.6 | 37 ± 14.4 |
| Male (%) | 326 (63%) | 6 (54%) | 320 (63%) |
| Language: | | | |
| Hebrew | 227 (44%) | 6 (54%) | 221 (43%) |
| English | 293 (56%) | 5 (46%) | 288 (57%) |

Reference is now made to FIG. 6. The predictive accuracy of the COVID-19 biomarker is shown and compared to COVID-19 positivity on the symptoms test set. The sensitivity of the biomarker was 54.5% [95% CI: 23.4-83.2%], specificity was 76% [95% CI: 72-80%], positive predictive value was 4.7% [95% CI: 2.73-7.94%] and negative predictive value was 98.72% [95% CI: 97.6-99.3%]. The numbers in parentheses represent fever prediction accuracy.

As described previously, the first 10 seconds of continuous speech in each recording were used in the voice feature extraction process followed by the support vector machine classifier that was optimized on the new training cohort (Table 4).

To compare the biomarker with fever screening, we labelled participants with fever (self-reported) as being COVID-19 positive. The results of this comparison are noted in parentheses in FIG. 6. The sensitivity of the fever screening was 18.2% [95% CI: 2.3-51.8%], specificity was 91.2% [95% CI: 88.3-93.5%], positive predictive value was 4.3% [95% CI: 1.2-13.8%] and negative predictive value was 98.1% [95% CI: 97.5-98.5%].

### Discussion and Implications

This study demonstrated an association between a non-invasive vocal biomarker and the presence of COVID-19. Data from 953 participants (78 COVID-19 positive and 875 negative) came from various recording devices (smartphones, computers and tablets) in diverse natural environments. To demonstrate the capability of the vocal biomarker, we built a balanced dataset (n=156) and an AUC of 69% was achieved, indicating that there is a unique vocal biomarker for COVID-19.

We next evaluated the ability of the biomarker to run on free speech recordings. For this we created a new training set of scripted speech recordings and a test set of free speech recordings. This analysis reached a sensitivity of >50% and specificity of −80%, strengthening the applicability of this biomarker to the general population in natural environments using spontaneous free speech.

Finally, we compared the biomarker to the widely used screening tool of temperature/fever. The biomarker demonstrated much higher sensitivity than fever screening (>50% vs. 18%), albeit with lower specificity (76% vs. 91%), indicating that the vocal biomarker prediction is at least as good as fever screening and outperforms fever in detecting COVID-19 positive individuals in this small sample size.

Vocal screening for COVID-19 has the potential to accelerate global efforts to recover from the pandemic.

The results presented here support the use of the Vocalis Health vocal biomarker as a first-line COVID-19 risk screening tool. It provides a non-invasive way to assess the general population for return to normal activities relying on voice signals that are accessible, cost-effective and do not require invasive tests.

While one or more embodiments of the invention have been described, various alterations, additions, permutations and equivalents thereof are included within the scope of the invention.

In the description of embodiments, reference is made to the accompanying drawings that form a part hereof, which show by way of illustration specific embodiments of the claimed subject matter. It is to be understood that other embodiments may be used and that changes or alterations, such as structural changes, may be made. Such embodiments, changes or alterations are not necessarily departures from the scope with respect to the intended claimed subject matter. While the steps herein may be presented in a certain order, in some cases the ordering may be changed so that certain inputs are provided at different times or in a different order without changing the function of the systems and methods described. The disclosed procedures could also be executed in different orders. Additionally, various computations that are herein need not be performed in the order disclosed, and other embodiments using alternative orderings of the computations could be readily implemented. In addition to being reordered, the computations could also be decomposed into sub-computations with the same results.

### BIBLIOGRAPHY

Bai Y, Yao L, Wei T, et al. “Presumed Asymptomatic Carrier Transmission of COVID-19.” JAMA 2020.
Bonneh, Yoram S., et al. “Abnormal speech spectrum and increased pitch variability in young autistic children.” Frontiers in Human Neuroscience 4, 2011; 237.
Bwire, George M.; Paulo, Linda S. “Coronavirus disease-2019: is fever an adequate screening for the returning travelers?” Tropical Medicine and Health, 2020, 48.1: 1-3.
Goldshtein, Evgenia, Ariel Tarasiuk, and Yaniv Zigel. “Automatic detection of obstructive sleep apnea using speech signals. IEEE Transactions on biomedical engineering 58.5 (2010): 1373-1382.”
Guan, Wei-jie, et al. “Clinical characteristics of coronavirus disease 2019 in China.” New England Journal of Medicine, 2020.
Guo, Li, et al. “Profiling early humoral response to diagnose novel coronavirus disease (COVID-19).” Clinical Infectious Diseases, 2020.
Kumar, Anurag, Maksim Khadkevich, and Christian Fügen. “Knowledge transfer from weakly labeled audio using convolutional neural network for sound events and scenes.” 2018 IEEE International Conference on Acoustics, Speech and Signal Processing (ICASSP). IEEE, 2018.
L, Yan; Xia, Liming. “Coronavirus disease 2019 (COVID-19): role of chest CT in diagnosis and management.” American Journal of Roentgenology, 2020, 1-7.
Maor, Elad, et al. “Vocal Biomarker is Associated with Hospitalization and Mortality among Heart Failure Patients.” Submitted to Journal of American Heart Association, 2019.
Richardson, Safiya, et al. “Presenting Characteristics, Comorbidities, and Outcomes Among 5700 Patients Hospitalized With COVID-19 in the New York City Area.” JAMA.
Rothe C, Schunk M, Sothmann P, et al., “Transmission of 2019-nCoV Infection from an Asymptomatic Contact in Germany.” N Engl J Med 2020.
Sara, Jaskanwal Deep Singh et al. “Non-invasive vocal biomarker is associated with pulmonary hypertension.” PLOS One, vol. 15, 4 e0231441. 16 Apr. 2020, doi:10.1371/journal.pone.0231441.
Uma Rani K., Holi M. S. “Automatic detection of neurological disordered voices using Mel cepstral coefficients and neural networks.” IEEE Point-of-Care Healthcare Technologies (PHT):76-79. Bangalore, India: Jan. 16-18, 2013.
World Health Organization. Coronavirus disease 2019 (COVID-19) Situation Report—97. 2020
Yu P, Zhu J, Zhang Z, Han Y, Huang L., “A familial cluster of infection associated with the 2019 novel coronavirus indicating potential person-to-person transmission during the incubation period.” J Infect Dis 2020.

1. A computer-based method for screening unknown subjects for COVID-19, comprising steps of:
recording at least one voice clip from a screened subject;
pre-processing the screened subject voice clip;
computing a spectrogram of the pre-processed screened subject voice clip;
extracting a feature vector from said screened subject spectrogram;
applying a machine learning classifier of a COVID-19 vocal biomarker on the extracted screened subject feature vector, thereby receiving a COVID-19 vocal biomarker value; and
outputting that the screened subject is COVID-19 positive or COVID-19 negative, based on the COVID-19 vocal biomarker value;
wherein the step of extracting the feature vector employs a pre-trained deep convolutional neural network (CNN).

2. The method of claim 1, wherein recording said at least one voice clip is made at a sampling rate of 16 kHz, 32 kHz, 44.1 kHz.

3. The method of claim 1, further comprising selecting one or more of the speech clips from a fixed time interval of continuous speech within an extended recording of one or more of the subjects.

4. The method of claim 1, wherein the pre-processing of said at least one voice clip comprises one or more steps selected from a group consisting of normalizing, down-sampling, and any combination thereof.

5. The method of claim 1, wherein the computing of the spectrograms is made with an algorithm selected from a group comprising a short-time Fourier transform (STFT), a fast Fourier transform (FFT), Mel spectrogram, or any combination thereof.

6. The method of claim 1, wherein the feature vectors each comprise 512 or 1024 dimensions.

7. The method of claim 1, further comprising steps for training said COVID-19 vocal biomarker, comprising
a. recording at least one voice clip from each subject in a cohort, each cohort subject having a known status of either COVID-19 positive and COVID-19;
b. pre-processing the cohort subject voice clips;
c. computing a spectrogram of each of the pre-processed cohort subject voice clips;
d. extracting feature vectors from each cohort subject spectrogram, using said CNN; and
e. training a machine classifier with said cohort subject feature vectors and said cohort subjects' known COVID-19 statuses, thereby producing said COVID-19 vocal biomarker.

8. The method of claim 7, further comprising a step of cross-validating models for developing said classifier.

9. The method of claim 8, further comprising a step of selecting one or more of said models with the highest areas-under-curve (AUCs) of a receiver operating curve (ROC) of each cross-validated model.

10. The method of claim 7, wherein the cohort subject voice clips comprise both scripted speech clips and free speech clips.

11. A computer-based system for screening unknown subjects for COVID-19, comprising
a. a recording module, configured to record at least one voice clip from a screened subject;
b. a pre-processing module, configured to pre-process the screened subject voice clip;
c. a spectrography module, configured to compute a spectrogram of the pre-processed screened subject voice clip;
d. a feature-extraction module, configured to extract a feature vector from said screened subject spectrogram;
e. a classification module, configured to apply a machine learning classifier of a COVID-19 vocal biomarker on the extracted screened subject feature vector, thereby receiving a COVID-19 vocal biomarker value; and
f. an output module, configured to output that the screened subject is COVID-19 positive or COVID-19 negative, based on the COVID-19 vocal biomarker value;
wherein the step of extracting the feature vector employs a pre-trained deep convolutional neural network (CNN).

12. The system of claim 11, wherein recording said at least one voice clip is made at a sampling rate of 16 kHz, 32 kHz, 44.1 kHz.

13. The system of claim 11, wherein the recording module is further configured to select one or more of the speech clips from a fixed time interval of continuous speech within an extended recording of one or more of the subjects.

14. The system of claim 11, wherein the pre-processing is further configured to pre-process by normalizing, down-sampling, or any combination thereof.

15. The system of claim 11, wherein the computing of the spectrograms is made with an algorithm selected from a group comprising a short-time Fourier transform (STFT), a fast Fourier transform (FFT), Mel spectrogram, or any combination thereof.

16. The system of claim 11, wherein the feature vectors each comprise 512 or 1024 dimensions.

17. The system of claim 11, further comprising a training module for training said COVID-19 vocal biomarker, said training module configured to
a. record at least one voice clip from each subject in a cohort, each cohort subject having a known status of either COVID-19 positive and COVID-19;
b. pre-process the cohort subject voice clips;
c. compute a spectrogram of each of the pre-processed cohort subject voice clips;
d. extract feature vectors from each cohort subject spectrogram, using said CNN;
e. train a machine classifier with said cohort subject feature vectors and said cohort subjects' known COVID-19 statuses, thereby producing said COVID-19 vocal biomarker.

18. The system of claim 17, further configured to cross-validate models for developing said classifier.

19. The system of claim 18, wherein the training module is further configured to select one or more of said models with the highest areas-under-curve (AUCs) of a receiver operating curve (ROC) of each cross-validated model.

20. The system of claim 11, wherein the cohort subject voice clips comprise both scripted speech clips and free speech clips.
